(19) United States
(12) Patent Application Publication
(10) Pub. No.: US 2006/0018870 A1
(43) Pub. Date: Jan. 26, 2006

(54) Rigid polymer and method of making same

(75) Inventors: Naoto Kuriyama, Aichi (JP); Mitsuru Akashi, Osaka (JP); Tatsuo Kaneko, Osaka (JP); Michiya Matsusaki, Osaka (JP); Tran Thi Hang, Phu Tho (VN)

Correspondence Address: POSZ LAW GROUP, PLC, 12040 SOUTH LAKES DRIVE, SUITE 101, RESTON, VA, 20191, US

(73) Assignees: TOYODA GOSEI CO., LTD., Nishikasugai-gun (JP); Mitsuru AKASHI, Suita-shi (JP)

(21) Appl. No.: 11/191,263

(22) Filed: Jul. 28, 2005

(30) Foreign Application Priority Data
Jul. 29, 2004 (JP) 2004-221214
Jun. 14, 2004 (JP) 2005-173896

(51) Int. Cl. A61K 31/74; C08H 1/06

(52) U.S. Cl. 424/78.01; 527/100

(57) ABSTRACT

A rigid polymer has: a homopolymer of one of a polymerizable bio-derived rigid compound having two or more reactive functional groups, a derivative of the bio-derived rigid compound, a hydrolyzed substance of the bio-derived rigid compound, a derivative of the hydrolyzed substance of the bio-derived rigid compound and the substances extracted from the bio resources, or copolymers of two or more of these compound, derivatives, and substances.

The present application is based on Japanese patent application Nos. 2004-221214 and 2005-173896, the entire contents of which are incorporated herein by reference.

### BACKGROUND OF THE INVENTION

1. Field of the Invention

The invention relates to a rigid polymer made from bio-derived rigid compound, bio-derived rigid compound derivative, or a substance of hydrolyzed bio-derived rigid compound and a method of making the same and, particularly, to a rigid polymer usable in a wide range of fields of electronic/electric parts, automotive parts, or the like and a method of making the same.

2. Description of the Related Art

Presently, polylactic acid has been used practically as a bio-derived polymer (e.g., reference to “Biodegradable Plastics Handbook”, edited by Yoshiharu DOI, first edition, p. 576-581, NTS Co., Ltd., May 26, 1995).

The polylactic acid is produced from starch obtained from plant resources such as corn and sweet potato and when discarded, it is decomposed to water and carbon dioxide by hydrolysis and biodegradation. The polylactic acid has been used as films for agriculture, disposable containers, and stationary.

However, conventional polymers produced from lactic acid have a thermal deformation temperature as low as about 60° C. and therefore it is impossible to use the polymers for parts of automotive interior parts and exterior parts, and parts of engine systems, which are required to have heat resistance and mechanical strength.

On the other hand, as polymer materials having heat resistance and mechanical strength, rigid polymers have been known well. Since the rigid polymers are polymers with high aromatic ring concentration, their molecules become rigid and excellent in heat resistance and mechanical strength.

### SUMMARY OF THE INVENTION

It is an object of the invention to provide a rigid polymer using bio-derived rigid compound, bio-derived rigid compound derivative, or a substance of hydrolyzed bio-derived rigid compound and usable in a wide range of fields of electronic/electric parts, automotive parts, or the like and a method of making the same.

(1) According to one aspect of the invention, a rigid polymer comprises:

a homopolymer of one of a polymerizable bio-derived rigid compound having two or more reactive functional groups, a derivative of the bio-derived rigid compound, a hydrolyzed substance of the bio-derived rigid compound, a derivative of the hydrolyzed substance of the bio-derived rigid compound and the substances extracted from the bio resources; or

copolymers of two or more of these compound, derivatives, and substances.

In this connection, the “rigid”polymer means a homopolymer or a copolymer having at least one rigid structural portion of an aromatic ring, an alicyclic ring, a double bond and a triple bond.

The homopolymer is preferable to comprise one of chlorogenic acids, chlorogenic acid derivatives, substances of hydrolyzed chlorogenic acids, derivatives of the hydrolyzed substances and substances extracted from bio-resources.

The copolymer is preferable to comprise one of chlorogenic acids, chlorogenic acid derivatives, substances of hydrolyzed chlorogenic acids, derivatives of the hydrolyzed substances and substances extracted from bio-resources, and 4-hydroxycinnamic acid or polylactic acid.

(2) According to another aspect of the invention, a production method of a rigid polymer comprising one of a bio-derived rigid compound, a derivative of the bio-derived rigid compound, a hydrolyzed substance of the bio-derived rigid compound, a derivative of the hydrolyzed substance, or a substance extracted from a bio-resource comprises steps of:

acetylating one of the polymerizable bio-derived rigid compound having two or more reactive functional groups, the derivative of the bio-derived rigid compound having two or more reactive functional groups, the hydrolyzed substance of the bio-derived rigid compound having two or more reactive functional groups, the derivative of the hydrolyzed substance having two or more reactive functional groups and the substance extracted from the bio-resource having two or more reactive functional groups; and

homo-polymerizing the acetylated compound or substance in the presence of a prescribed catalyst.

(3) According to another aspect of the invention, a production method of a rigid polymer by copolymerizing one of a first bio-derived rigid compound, a derivative of the first bio-derived rigid compound, a hydrolyzed substance of the first bio-derived rigid compound, a derivative of the hydrolyzed substance of the first bio-derived rigid compound and a first substance extracted from a bio-resource with one of a second bio-derived rigid compound, a derivative of the second bio-derived rigid compound, a hydrolyzed substance of the second bio-derived rigid compound, a derivative of the hydrolyzed substance of the second bio-derived rigid compound and a second substance extracted from a bio-resource comprises steps of:

acetylating a first material comprising one of the polymerizable first bio-derived rigid compound having two or more reactive functional groups, the derivative of the first bio-derived rigid compound, the hydrolyzed substance of the first bio-derived rigid compound, the derivative of the hydrolyzed substance of the first bio-derived rigid compound and the first substance extracted from the bio-resource as well as a second material comprising one of the polymerizable second bio-derived rigid compound having two or more reactive functional groups, the derivative of the second bio-derived rigid compound, the hydrolyzed substance of the second bio-derived rigid compound, the derivative of the hydrolyzed substance of the second bio-derived rigid compound and the second substance extracted from the bio-resource; and
copolymerizing the first acetylated material with the second acetylated material in the presence of a prescribed catalyst.

According to the invention, the homopolymer can be produced from one or more of, and while the copolymer can be produced from two or more of the polymerizable bio-derived rigid compounds having two or more reactive functional groups, derivatives of the bio-derived rigid compounds and hydrolyzed substances of the bio-derived rigid compounds, so that a rigid polymer having high beat resistance and processibility and usable in a wide range of fields of electronic/electric parts, automotive parts, or the like can be provided.

### BRIEF DESCRIPTION OF THE DRAWINGS

The preferred embodiments according to the invention will be explained below referring to the drawings, wherein:

FIG. 1 is a drawing showing the structure analysis of a copolymer of 4HCA and caffeic acid, a caffeic acid homopolymer, and a 4HCA homopolymer, which are rigid polymers according to the first and the sixth embodiments of the invention by Fourier transform infrared spectrophotometer;

FIG. 2 is a drawing showing the results of wide-angle x-ray diffractiometry of the copolymer of 4HCA and caffeic acid, the caffeic acid homopolymer, and the 4HCA homopolymer which are rigid polymers according to the first and the sixth embodiments of the invention;

FIG. 3 is a liquid crystal photograph of the copolymer of 4HCA and caffeic acid, which is a rigid polymer according to the first embodiment of the invention;

FIG. 4 is a drawing showing the result of differential scanning calorimetry (DSC) of the copolymer of 4HCA and caffeic acid which is a rigid polymer according to the first embodiment of the invention;

FIG. 5 is a drawing showing the structure analysis of a polymer of coffee bean extraction substance, which is a rigid polymer according to the seventh embodiment of the invention by Fourier transform infrared spectrophotometer;

FIG. 6 is a drawing showing the result of differential scanning calorimetry (DSC) of the copolymer of polylactic acid and caffeic acid, which is a rigid polymer according to the ninth embodiment of the invention;

FIG. 7 is a drawing showing that a produced polymer is finely granulated; and

FIG. 8 is a drawing tracing the structure alteration of the copolymer of 4HCA and caffeic acid by UV-Vis in the case UV rays are irradiated in various durations.

### DESCRIPTION OF THE PREFERRED EMBODIMENTS

### First Embodiment

A rigid polymer according to the first embodiment of the invention will be described. The rigid polymer is a copolymer of 4-hydroxycinnamic acid (4HCA) and caffeic acid and is defined by the following formula (1). Here, 4HCA is a polymerizable bio-derived rigid compound having two reactive functional groups and caffeic acid is a hydrolyzed substance of polymerizable bio-derived rigid compound having three reactive functional groups. 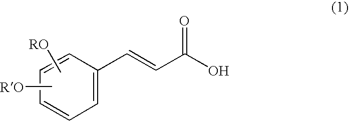
wherein R and R′ independently denote a polymer, H, or acetyl.

One example of a production method of the rigid polymer of the first embodiment will be described. At first 4HCA (defined by the following formula (2)) 0.82 g (5 mmol) and caffeic acid (defined by the following formula (3)) 1.7 g (10 mmol) are loaded to a four-neck flask and further acetic anhydride having a catalytic function (10 ml) and sodium acetate as an ester interchange catalyst are added. Next, the four-neck flask is purged with dry nitrogen and immersed in a silicon oil tank. Next, the four-neck flask is wrapped with an aluminum foil as a light shielding material so as to shield the light and in that condition, the silicon oil tank is heated to carry out polymerization. Next, the product of the polymerization reaction is extracted and dissolved in pentafluorophenol and the resulting solution is poured to ethanol to re-precipitate the product. Next, the precipitate is collected by filtration and subjected to impurity removal by methanol using a Soxhlet's extractor to obtain a copolymer. The copolymer of 4HCA and caffeic acid obtained in such a manner is vacuum dried and subjected to various measurements. In this connection, the production conditions are not limited to the above-mentioned manner and for example, the loading amounts, polymerization time, polymerization temperature, amounts of the catalyst and the solvent may be changed and operation of vacuum treatment during the polymerization and addition of a reinforcing material to the polymer may be carried out.

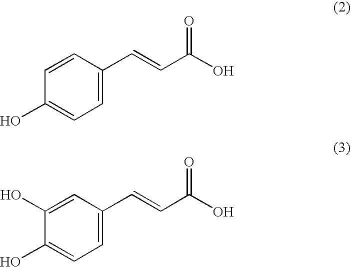

The rigid polymer according to the first embodiment is provided with the following characteristics and physical properties.

(1) Production Conditions

Table 1 shows molding results of the 4HCA-caffeic acid copolymer in respective production conditions. According to the respective production conditions, the molding is made possible by copolymerization and at the same time the toughness is improved and good molded bodies can be obtained. Further, if the polymerization temperature is increased, molding-possible temperature is further increased although it depends on the loading amount of the caffeic acid and at the same time the toughness is further improved and better molded bodies can be obtained. It is confirmed that the characteristics of the polymer are changed in accordance with the alteration of the solvent amount and the polymerization time. Accordingly, as the production conditions for tough copolymers, the composition ratio of the caffeic acid is preferably 20 to 75 mol %, the polymerization temperature is preferably 175 to 225° C., and the solvent amount and the polymerization time for the polymerization are preferable to be excess.

TABLE 1

| | 175° C. | 200° C. | 225° C. |
|---|---|---|---|
| 100/0 | X(200) | X(280) | X(295) |
| 75/25 | 6 h: Δ(110) | ◯(150) | 2.5 h: ◯(150)A |
| | 9 h: Δ(120) | | 2.5 h: ◯(225)AA |
| | | | 6 h: ◯(200) |
| | | | 6 h: ◯(230)A |
| 50/50 | Δ(120) | ◯(190) | 2.5 h: ◯(240)A |
| | | ◯(130)A | 6 h: X(210) |
| | | | 6 h: ◯(240)A |
| 25/75 | X(120) | Δ(190) | X(220) |
| 0/100 | X(110) | X(210) | — |
| 75/25(highly | 15 h: ◯(105) | ◯(165) | ◯(200) |
| excessive solvent) | | | |
| 50/50(highly | — | ◯(170) | ◯(210) |
| excessive solvent) | | ◯(210)※ | |
| 25/75(highly | — | Δ(175) | Δ(170) |
| excessive solvent) | | | |

<Other Polymer Production Conditions>
Polymerization time: 6 h (some of polymers are polymerized for 2.5 h, 9 h or 15 h)
Catalyst: a prescribed amount of sodium acetate
Solvent: a prescribed amount of acetic anhydride
Others/(※): polymerization in vacuum, (A): polymerization of acetylated monomer, (AA): polymerization of acetylated monomer and annealing
   <Molding Conditions/Results>
Molding temperature: written in parentheses
Molding time: heating for 8 minutes and cooling for 5 minutes
Molding results:
◯: good molding, Δ: partially cracked, v: very significantly cracked, and -: not molded
   (2) Chemical Structure

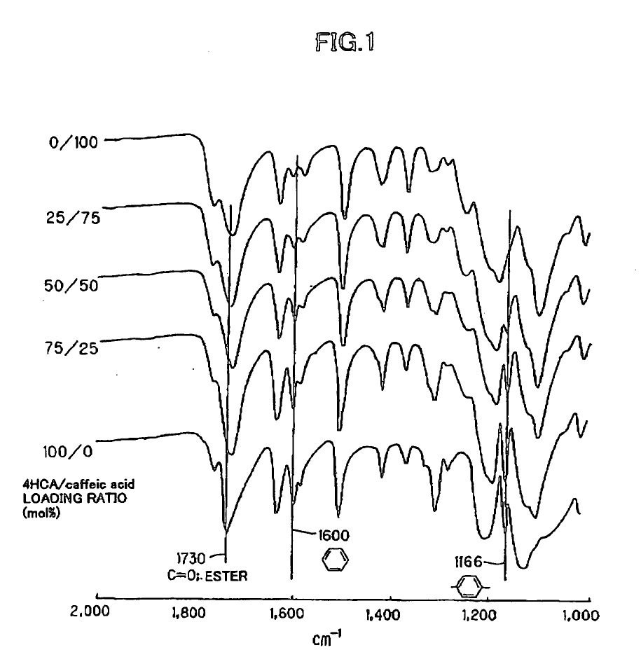

FIG. 1 is a drawing showing the structure analysis of a copolymer of 4HCA and caffeic acid, a caffeic acid homopolymer, and a 4HCA homopolymer by Fourier transform infrared spectrophotometer. According to this, no C═O at 1670 to 1640/cm⁻¹ peak attributed to caffeic acid appears and a peak at 1730/cm⁻¹ attributed to ester (C═O) appears. The peak intensity at 1600/cm⁻¹ attributed to an aromatic ring and the peak intensity at 1166/cm⁻¹ attributed to bi-substituted aromatic ring are changed corresponding to the composition ratio alteration of caffeic acid. The causes for the fact are that the peak at 1600/cm⁻¹ for the caffeic acid is weak and that no clear peak appears at 1166/cm⁻¹ since the caffeic acid comprises tri-substituted aromatic ring. Further, it can be assumed that copolymerized polymer is produced because the peak intensity is changed depending on the 4HCA/caffeic acid loading ratio.

(3) Results of Wide-Angle X-Ray Diffractiometry

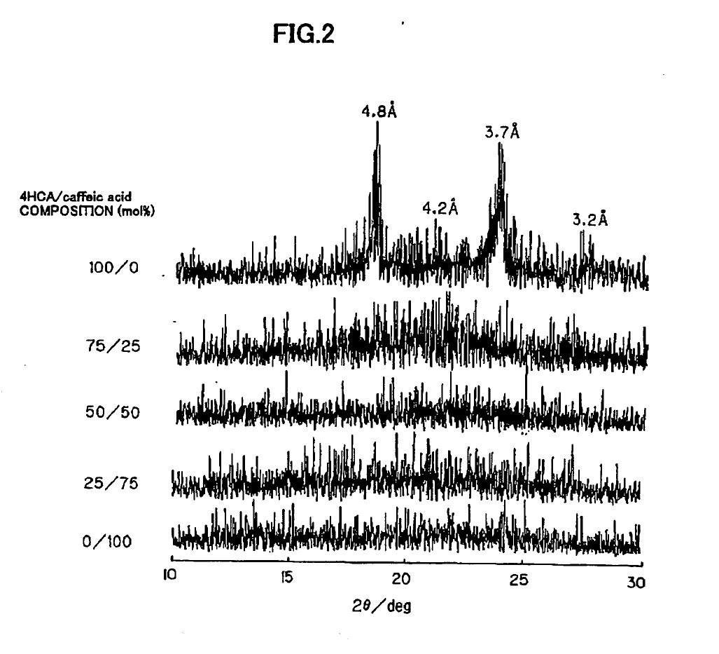

FIG. 2 is a view showing the results of wide-angle x-ray diffractiometry of the copolymer of 4HCA and caffeic acid, the caffeic acid homopolymer, and the 4HCA homopolymer. According to the results of the wide-angle x-ray diffractiometry, both peaks of 4.8 Å and 3.7 Å, which appear 4HCA composition ratio is 100%, disappear in the case of the polymers. The fact shows the crystallinity is lost by introduction of caffeic acid and it is assumed that the copolymer of 4HCA and caffeic acid is produced.

(4) Solubility

Table 2 shows the solubility of the rigid polymer according to the first embodiment. It is evaluation of the solubility of 4HCA-caffeic acid copolymers in various solvents in the case 4HCA-caffeic acid copolymers are obtained under condition of changing the loading ratios of 4HCA and caffeic acid and the polymerization temperature and the polymers axe immersed in the various solvents. The copolymerization is carried out at 175° C., 185° C., and 200° C.

TABLE 2

| | SYNTHESIS CONDITONS | | | | | | | | | | | | | |
|---|---|---|---|---|---|---|---|---|---|---|---|---|---|---|
| | | | 4HCA/CAFFEIC ACID LOADING RATIO (mol %) | | | | | | | | | | | |
| | | | | | | | | polymer- | | | | | | |
| | | | | | | | | ization | | | | | | |
| | | | polymerization | | | | | temp = | | polymerization | | | | |
| | (MONOMER) | | temp = 175° C. | | | | | 185° C. | | temp = 200° C. | | | | |
| SOLVENTS | 4HCA | caffeic acid | 100/0 | 95/5 | 80/20 | 50/50 | 0/100 | 80/20 | 50/50 | 100/0 | 75/25 | 50/50 | 25/75 | 0/100 |
| WATER | ◯※ | ◯※ | X | X | X | X | X | X | X | X | X | X | X | X |
| METHANOL | ◯ | ◯ | X | X | X | X | X | X | X | X | X | X | X | X |
| ACETONE | ◯ | ◯ | X | X | X | X | ◯ | X | X | X | X | X | X | X |
| ACETONITRILE | ◯ | ◯ | X | X | X | X | Δ | X | ◯ | X | X | X | X | X |
| CHLOROFORM | X | X | X | X | Δ | ◯ | ◯ | Δ | ◯ | X | X | X | X | X |
| TFA/DCM 1/5 | ◯ | ◯ | — | — | — | — | — | — | — | ◯ | ◯ | X | X | X |
| V/V % | | | | | | | | | | | | | | |
| TOLUENE | X | X | X | X | X | X | X | X | X | X | X | X | X | X |
| HEXANE | X | X | X | X | X | X | X | X | X | X | X | X | X | X |
| NMP | ◯ | ◯ | — | — | — | — | — | — | — | X | ◯ | ◯ | ◯ | ◯ |
| DMSO | ◯ | ◯ | X | Δ | X | ◯※ | ◯ | X | ◯※ | X | X | X | X | X |
| DMF | ◯ | ◯ | Δ | Δ | ◯ | ◯ | ◯ | Δ | ◯※ | X | X | Δ | Δ | ◯ |
| THF | ◯ | ◯ | X | Δ | X | Δ | ◯ | X | X | X | X | X | X | X |
| PENTAFLUOROPHENOL | — | — | ◯ | ◯ | ◯ | ◯ | ◯ | ◯ | ◯ | ◯ | ◯ | ◯ | ◯ | ◯ |

◯: soluble
Δ: partially soluble
X: insoluble
◯※: soluble when heated

According to Table 2, it is found that the solubility of the produced rigid polymers differs from that of the monomers and also differs depending on the production conditions, proving formation of the rigid polymers.

(5) Molecular Weight Measurement

Table 3 shows the measurement results of the molecular weights of the rigid polymers according to the first embodiment. As a measurement facility, HLC-8120GPC manufactured by TOSOH is used and DMF is a solvent used and the solvent flow speed is 0.6 ml/min. According to the result, polymers are partially hardly soluble in DMF and measurement is thus difficult and the measurement values of some polymers are less reliable, however it is clear that the molecular weight is changed depending on the production method conditions and high molecular weight polymers are obtained.

TABLE 3

| POLIMERIZATION | POLIMERIZATION | | |
|---|---|---|---|
| COMPOSITION | TEMP | | |
| (4HCA/caffeic acid) | 175° C. | 200° C. | 225° C. |
| 100/0 | 1600※ | — — | — — |
| 75/25 | 6 h: 5600 | — — | — — |
| | 9 h: 12000※ | | |
| 50/50 | 5100 | 32000※ | 14000※ |
| 25/75 | 11000 | 45000※ | 11000※ |
| 0/100 | 13000 | 75000 | — |
| 75/25(highly | — | 45000 | — |
| excessive solvent) | | | |
| 50/50(highly | — | 91000 | — |
| excessive solvent) | | | |
| 25/75(highly | — | 80000 | — |
| excessive solvent) | | | |

<Explanation of results>
—: measurment not done
— —: measurment impossible owing to insolubility in solvent
※: measurment in partially dissolved state in solvent

(6) Liquid Crystallinity

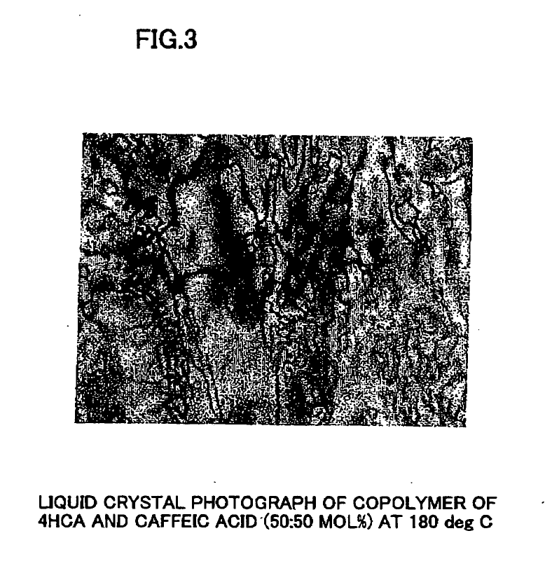

FIG. 3 shows a photograph of the 4HCA-caffeic acid copolymer (composition 50 mol %: 50 mol %) heated and taken by a deflection microscope. It is confirmed that the 4HCA-caffeic acid copolymer (composition 50 mol %: 50 mol %) has the nematic liquid crystal characteristic since schlieren structure is observed when the polymer is heated.

(7) Calorimetry

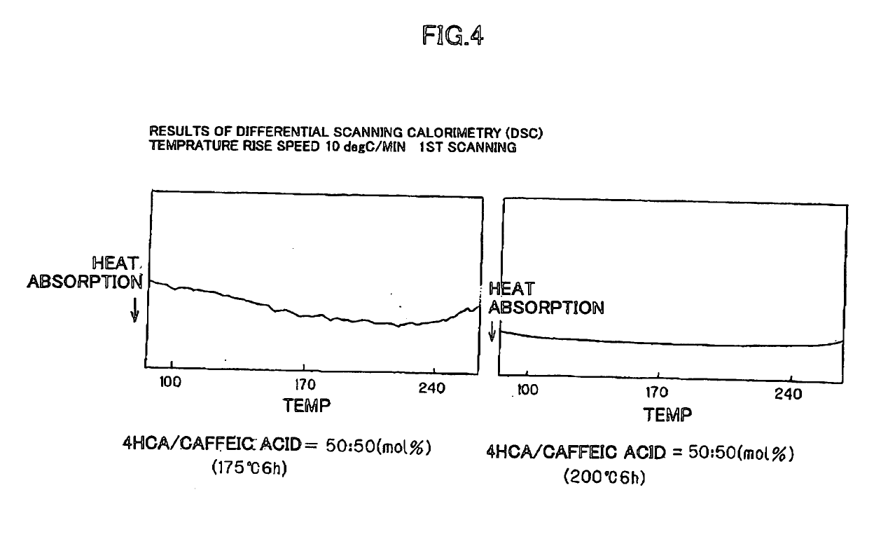

FIG. 4 shows parts of the results of differential scanning calorimetry (DSC) in the case of increasing the temperature from a room temperature to 300° C. at the rate of 10° C./min. Since both of the 4HCA-caffeic acid copolymer (composition 50 mol %: 50 mol %) obtained by reaction at 175° C. for 6 hours and 4HCA-caffeic acid copolymer (composition 50 mol %: 50 mol %) obtained by reaction at 200° C. for 6 hours have high glass transition point, they may be used for electronic/electric parts.

(8) Flexural Test

The flexural test is carried out by two type techniques.

(i) Flexural Test Based on JIS K 7171

The 4HCA-caffeic acid copolymer (composition 50 mol % 50 mol %) obtained by reaction at 175° C. for 6 hours is preliminarily dried at 80° C. for 1 hour. Next, pressure of 30 kg/cm2 is applied at 185° C. for 6 minutes to obtain a molded product in form of a plate with a size of 200 mm×200 mm×3 mm thickness, which is cut to obtain a sample for measurement. Evaluation is carried out according to JIS K7171 at 23° C., 48 mm supporting point distance, and 1.5 mm/min testing speed using Universal Electromechanical Materials Test Machine 5528 manufactured by Instron Corporation is used as the evaluation facility.

(ii) Flexural Test Using Small Type Material Tester

The respective polymers produced by the different production conditions shown in Table 1 are compressively molded to small pieces with 6 mm×50 mm×3 mm thickness by molding conditions shown in Table 1 to obtain samples for measurement. Evaluation is carried out at 23° C. and 0.1 mm/min testing speed using a testing tool comprising a support with 2 mm R, a presser with 5 mm R and having a supporting point distance 29 mm a small type material tester manufactured by Imoto Machinery Co., Ltd. with reference to JIS K 7171.

(9) Compression Test

The compression test is carried out as follows for 4HCA-caffeic acid copolymers with different compositions obtained by reaction at 200° C. for 6 hours.

(i) Production of Test Pieces

Molding is carried out at 250° C. to obtain compression measurement.

(ii) Evaluation facility

The evaluation is carried out by AGS-H/EZ series manufactured by Shimadzu Corporation

### Effects of the First Embodiment

The following effects can be obtained by the rigid polymers according to the first embodiment.
(A) Copolymers of 4HCA and caffeic acid show nematic liquid crystallinity when heated and therefore, they can be classified and used as liquid crystal polymers.
(B) Copolymers of 4HCA and caffeic acid can be molded in form of copolymers and at the same time, they become tough and provide good molded bodies. In the case the polymerization temperature is controlled to be high, although depending on the loading amount of the caffeic acid, the molding possible temperature is increased more and thereby the strength is increased more and better molded bodies can be obtained. The polymer properties are changed by changing the composition, polymerization temperature and polymerization time in such a manner and the polymer properties are confirmed to change also depending on alteration of the solvent amount and conduction of refining as well as a operation such as polymerization in vacuum or annealing. Depending on the conditions, elastic properties may be obtained. Accordingly, the conditions for producing tough copolymers are preferably as follows: the composition ratio of caffeic acid is preferably 20 to 75 mol %; the polymerization temperature is preferably 175 to 225° C., and the solvent amount and the polymerization duration at the time of polymerization are preferably in excess. In such a manner, the moldability, heat resistance, and dynamic strength of the polymers can be changed by changing the production conditions of the polymers.
(C) Since 4HCA and caffeic acid are soluble in various solvents, the options of processing methods of their polymers is increased, e.g. thin film formation by wet spinning and casting and fine granulation, are made possible.
(D) Since 4HCA and caffeic acid are soluble in various solvents, various physical properties measurement and structure analysis, e.g. molecular weight analysis and optical reactivity, are made possible,
(E) It is possible to produce polymers with various molecular weights from 4HCA and caffeic acid depending on the production conditions.
(F) Based on the results of the flexural test according to JIS K 7171, flexural modulus is 2.93 GPa. Table 4 shows the flexural test results of the polymers with different production conditions by the small type material tester. From the results, flexural physical properties are found differing depending on the polymer production method conditions. Further, it is found that there is a close correlation that these polymers with high physical properties have high molecular weights and the polymers can be processed to be fibers, so that they can be used fillers.
(G) Table 5 shows the results of compression test. Compressive strength of copolymers of 4HCA and caffeic acid is in a range of 30 to 100 MPa and compressive modulus of elasticity of them is in a range of 0.6 to 1.6 GPa, although depending on the composition and they can be used for automotive parts and electronic/electric parts.

(H) The measurement by the flexural test by the small type material tester shown in Table 4 and the results of the compression test shown in Table 6 are not by testing methods in compliance with JIS and therefore the results cannot be compared with the results of tests in compliance with JIS and only for investigations of conditions for physical property improvement. Further, since the physical properties may change also depending on the sizes of the molded bodies and methods for molding, for example, molding temperature and time, the values shown in Table 4 and Table 5 are not limited as shown,

TABLE 4

| | 175° C. | 200° C. | 225° C. |
|---|---|---|---|
| 100/0 | — | — | — |
| 75/25 | 6 h: — | 9(1370) | 2.5 h: 17(2400)A |
| | 9 h: 16(2120) | 17(2100)R | 2.5 h: 34(3170)AA |
| | | | 6 h: 16(1940) |
| | | | 6 h: 22(1310)A |
| | | | 6 h: 31(2500)R |
| 50/50 | 5(820) | 12(1840) | 2.5 h: 14(1800)A |
| | | 18(2410)R | 6 h: 33(3020)A |
| | | 15(1900)A | |
| 25/75 | — | 7(1260) | — |
| | | 10(1820)R | |
| 0/100 | — | X | — |
| 75/25 (highly | 15 h: 9(1340) | 10(1800) | 24(2950) |
| excessive solvent) | | 13(2020)R | |
| 50/50(highly | — | 16(2250) | 25(2630) |
| excessive solvent) | | 31(3210)R | 32(2420)R |
| | | 21(2490)※ | |
| 25/75(highly | — | 10(1680) | 8(1500) |
| excessive solvent) | | | |

<Meaning of symbols>
—: measurment not performed
R: refined polymer
A: polymer produced from acetylated monomer
※: polymer by vacuum polymerization

TABLE 5

| | COMPRESSIVE | COMPRESSIVE |
|---|---|---|
| 4HCA/CAFFEIC ACID | STRENGTH | MODULUS |
| LOADING RATIO (mol %) | (MPa) | ELASTICITY (GPa) |
| 25/75 | 103 | 1.6 |
| 50/50 | 95 | 1.7 |
| 75/25 | 31 | 0.6 |

Additionally, in place of the above-mentioned 4HCA and caffeic acid, a polymerizable bio-derived rigid compound having two or more reactive functional groups, a derivative of the polymerizable bio-derived rigid compound having two or more reactive functional groups, a hydrolyzed substance of the polymerizable bio-derived rigid compound having two or more reactive functional groups, a polymerizable bio-derived rigid compound having three or more reactive functional groups, a derivative of the polymerizable bio-derived rigid compound having three or more reactive functional groups, or a hydrolyzed substance of the polymerizable bio-derived rigid compound having three or more reactive functional groups may be used in combination. That is, a bio-derived rigid compound, a derivative of the bio-derived rigid compound, and a hydrolyzed substance of the bio-derived rigid compound may be used in combination so as to make polymers to be produced have bonding groups such as ester group, thioester group, amino group, thioamido, ketone group, thioketo group, carbonate group, thiocarbonate group, urethane group, thiourethane group, imido group, thioimido group, isoimido group, ether group, thioether group, amine group, azomethine group, azo group, hydroxamic acid group, acid anhydride, oxazole group, isooxazole group, thiazole group, imidazole group, oxadiazole group, triazole group, triazine group, imidazole group, hydantoin group, pyrazole group, oxazinone group, quinazolone group, quinazolinedione group, quinoxaline group, and phthalazinone group.

Further, the polymers of the invention may include copolymers of the polymerizable bio-derived rigid compound having two or more reactive functional groups and one or more polymerizable compounds having three or more reactive functional groups and a rigid structural portion such as an alicyclic structure, a double bond, a triple bond or the like and selected from nucleic acids, amino acids, saccharides, fatty acids, terpenes, porphyrins, flavonoids, steroids, chlorogenic acids, and alkaloids and copolymers of the polymerizable bio-derived rigid compound having two or more reactive functional groups and two or more of polymerizable compounds having three or more reactive functional groups and a rigid structural portion such as an alicyclic structure, a double bond, a triple bond or the like and selected from nucleic acids, amino acids, saccharides, fatty acids, terpenes, porphyrins, flavonoids, steroids, chlorogenic acids, and alkaloids.

### Second Embodiment

Next, a rigid polymer according to the second embodiment of the invention will be described. The rigid polymer is a copolymer of 4-hydroxycinnamic acid (4HCA) and caffeic acid and is different from the rigid polymer of the first embodiment in the production method.

(Production Method)

At first, 4HCA or caffeic acid is loaded to a four-neck flask and in the presence of acetic anhydride, the flask is purged with nitrogen and reaction of each compound is carried out at 165° C. for 3 hours to produce acetylated 4HCA or acetylated caffeic acid, which is refined thereafter. Next, the obtained aceylated 4HCA and acetylated caffeic acid is polymerized, for example, at 195° C. for 6 hours after purging with nitrogen to obtain a copolymer. The copolymer of 4HCA and caffeic acid obtained in such a manner is subjected to various measurements. In this connection, the production conditions are not limited to the above-mentioned manner and for example, the loading amounts, polymerization time, polymerization temperature, amounts of the catalyst and the solvent may be changed and operation of vacuum treatment during the polymerization and addition of a reinforcing material to the polymer may be carried out.

Table 1 shows molding results of the acetylated 4HCA-acetylated caffeic acid copolymer in respective production conditions. According to the respective production condition, the molding is made possible by using acetylated monomer and the toughness is improved and good molded bodies can be obtained.

Table 4 shows that the polymers using acetylated monomer have good flexural physical property and if it is annealed, the flexural physical property is better. So, the polymers can be processed to be a fiber and they can be used as fillers.

Table 6 shows the solubility of the rigid polymer in various solvents. Since the solubility is changed from those of the acetylated monomers, it is supposed that 4HCA-caffeic acid copolymer is produced. Additionally, in place of acetic anhydride, trifluoroacetic anhydride may be used in the acetylation step.

TABLE 6

| | SYNTHESIS CONDITIONS | | | | |
|---|---|---|---|---|---|
| | | | 4HCA/CAFFEIC | | |
| | (ACETYLATED | | ACID LOADING | | |
| | MONOMER) | | RATIO (mol %) | | |
| SOLVENTS | 4HCA | caffeic acid | 100/0 | 50/50 | 0/100 |
| WATER | X | X | X | X | X |
| METHANOL | ◯※ | ◯ | X | X | X |
| ACETONE | ◯ | ◯ | Δ | X | X |
| ACETONITRILE | Δ | ◯ | Δ | X | X |
| CHLOROFORM | Δ | ◯ | X | X | X |
| TFA/DCM 1/5 | — | — | — | — | — |
| V/V % | | | | | |
| TOLUENE | X | X | X | X | X |
| HEXANE | X | X | X | X | X |
| NMP | — | — | — | — | — |
| DMSO | ◯ | ◯ | ◯※ | X | Δ |
| DMF | ◯ | ◯ | ◯ | X | X |
| THF | ◯ | ◯ | X | X | X |

◯: soluble
Δ: partially soluble
X: insoluble
◯※: soluble when heated
—: not synthesized

### Effects of the Second Embodiment

According to the second embodiment, even if the production method is changed, the rigid polymer excellent in heat resistance and processibility can be produced. The molecular weight can be increased by using trifluoroacetic anhydride in place of acetic anhydride.

### Third Embodiment

Next, a rigid polymer according to the second embodiment of the invention will be described. The rigid polymer is a copolymer of 4-hydroxycinnamic acid (4HCA) and chlorogenic acid and has permeability. Here, chlorogenic acid is a polymerizable bio-derived rigid compound having two or more reactive functional groups.

Table 7 shows solubility of 4HCA and chlorogenic acid in various solvents. According to this, the copolymer obtained by loading 4HCA monomer and chlorogenic acid monomer at each 50 mol % ratio into a reaction vessel, purging the vessel with nitrogen, and carrying out reaction at 175° C. for 6 hours has different solubility from that of poly (4HCA) in various solvents. The solubility of the copolymer also differs from that of each monomer.

TABLE 7

| SYNTHESIS CONDITIONS | | | |
|---|---|---|---|
| | LOADING RATIO (mol %) | | |
| (MONOMER) | | 4HCA | 4HCA |

| | | chlorogenic | quinic | | chlorogenic | quinic |
|---|---|---|---|---|---|---|
| SOLVENTS | 4HCA | acid | acid | poly(4HCA) | acid 50/50 | acid 50/50 |
| WATER | ◯※ | ◯ | ◯ | X | X | X |
| METHANOL | ◯ | ◯ | ◯ | X | X | X |
| ACETONE | ◯ | ◯ | X | X | ◯ | Δ |
| ACETONITRILE | ◯ | X | X | X | ◯ | Δ |
| TFA/DCM 1/5 V/V % | ◯ | — | — | — | — | — |
| TOLUENE | X | X | X | X | X | X |
| HEXANE | X | X | X | X | X | X |
| NMP | ◯ | — | — | — | — | — |
| DMSO | ◯ | ◯ | ◯ | X | ◯ | Δ |
| DMF | ◯ | ◯ | ◯ | X | ◯ | ◯ |
| THF | ◯ | ◯ | X | X | ◯ | ◯ |

◯: soluble
Δ: partially soluble
X: insoluble
◯※: soluble when heated
—: not synthesized

### Effects of the Third Embodiment

According to the third embodiment, rigid polymer excellent in solubility in solvents and processability to be formed into thin films can be obtained by reaction of 4HCA monomer and chlorogenic acid monomer.

### Fourth Embodiment

The rigid polymer according to the fourth embodiment of the invention will be described. The rigid polymer is a copolymer of 4HCA and quinic acid. Here, the quinic acid is a hydrolyzed substance of a polymerizable bio-derived rigid compound having two or more reactive functional groups.

Table 7 shows solubility of 4HCA and quinic acid in various solvents. According to this, the copolymer obtained by loading 4HCA monomer and quinic acid monomer at each 50 mol % ratio into a reaction vessel, purging the vessel with nitrogen, and carrying out reaction at 175° C. for 6 hours has changed the solubility from that of poly(4HCA), 4HCA monomer, and quinic acid monomer.

### Effects of the Fourth Embodiment

According to the fourth embodiment, rigid polymer excellent in solubility in solvents and processability to be formed into thin films can be obtained by reaction of 4HCA monomer and quinic acid monomer.

### Fifth Embodiment

Next, a rigid polymer according to the fifth embodiment of the invention will be described. The rigid polymer is a copolymer obtained by loading 4HCA monomer and coffee bean extraction substance at each 50 mol % ratio into a reaction vessel, purging the vessel with nitrogen, and carrying out reaction at 175° C. for 6 hours. Herein, as the coffee bean, Java Robusta coffee bean is used.

Table 8 shows solubility of the copolymer obtained by loading 4HCA monomer and coffee bean extraction substance monomer at each 50 wt. % ratio in respective solvents. According to this, the solubility of the copolymer of 4HCA monomer and coffee bean extraction substance monomer in respective solvents is changed from that of poly(4HCA), 4HCA monomer, or coffee bean extraction substance monomer.

TABLE 8

| | SYNTHESIS CONDITIONS | | | | |
|---|---|---|---|---|---|
| | | | LOADING RATIO | | |
| | (MONOMER) | | (wt. %) | | |
| | | cofee bean | 4HCA/coffee bean | | |
| | | extracted | extracted substance | | |
| SOLVENTS | 4HCA | substance | 100/0 | 50/50 | 0/100 |
| WATER | ◯※ | X | X | X | Δ |
| METHANOL | ◯ | ◯ | X | Δ | ◯ |
| ACETONE | ◯ | X | X | Δ | Δ |
| ACETONITRILE | ◯ | X | X | Δ | ◯ |
| CHLOROFORM | X | X | X | Δ | ◯ |
| TFA/DCM 1/5 V/V % | ◯ | — | — | — | — |
| TOLUENE | X | X | X | Δ | Δ |
| HEXANE | X | X | X | X | X |
| NMP | ◯ | — | — | — | — |
| DMSO | ◯ | ◯ | X | Δ | ◯ |
| DMF | ◯ | ◯ | X | ◯ | ◯ |
| THF | ◯ | X | X | ◯ | ◯ |

◯: soluble
Δ: partially soluble
X: insoluble
◯※: soluble when heated
—: not synthesized

### Effects of the Fifth Embodiment

According to the fifth embodiment, rigid polymer excellent in solubility in solvents and processability to be formed into thin films can be obtained by reaction of 4HCA monomer and the coffee bean extraction substance.

### Sixth Embodiment

Next, a rigid polymer according to the sixth embodiment of the invention will be described. The rigid polymer is a homopolymer obtained by loading caffeic acid monomer into a four-neck flask, purging the flask in the presence of acetic anhydride and sodium acetate, and carrying out reaction at a prescribed temperature for 6 hours.

Table 2 shows solubility of the caffeic acid homopolymer, the rigid polymer, in respective solvents. According to this, the solubility of caffeic acid and the solubility of the produced rigid polymer in respective solvents are changed.

### Effects of the Sixth Embodiment

According to the sixth embodiment, caffeic monomer is polymerized by the reaction to obtain a non-crystalline polymer as shown in FIG. 2. The solubility in the respective solvents and the processability can be changed depending on the production conditions. Further, as shown in Table 9, the obtained polymer shows compressive strength 105 MPa and compressive modulus of elasticity 1.9 GPa in the compression test and it proves that the polymer is tougher than poly(4HCA). Additionally, caffeic acid homopolymer can be produced by using solely acetylated caffeic acid monomer obtained in the second embodiment. In this case, as shown in Table 6, it is found that the solubility in the respective solvents changes.

TABLE 9

| 4HCA/CAFFEIC | | COMPRESSIVE |
|---|---|---|
| ACID LOADING | COMPRESSIVE | MODULUS |
| RATIO (mol %) | STRENGTH (MPa) | ELASTICITY (GPa) |
| 100/0 | 7 | 0.6 |
| 0/100 | 105 | 1.9 |

Additionally, in place of the above-mentioned caffeic acid, a polymerizable bio-derived rigid compound having three or more reactive functional groups, a derivative of the polymerizable bio-derived rigid compound having three or more reactive functional groups, or a hydrolyzed substance of the polymerizable bio-derived rigid compound having three or more reactive functional groups can be used. That is, a bio-derived rigid compound, a derivative of the bio-derived rigid compound, or a hydrolyzed substance of the bio-derived rigid compound may be used in combination so as to make polymers to be produced have bonding groups such as ester group, thioester group, amino group, thioamido, ketone group, thioketo group, carbonate group, thiocarbonate group, urethane group, thiourethane group, imido group, thioimido group, isoimido group, ether group, thioether group, amine group, azomethine group, azo group, hydroxamic acid group, acid anhydride, oxazole group, isooxazole group, thiazole group, imidazole group, oxadiazole group, triazole group, triazine group, imidazole group, hydantoin group, pyrazole group, oxazinone group, quinazolone group, quinazolinedione group, quinoxaline group, and phthalazinone group.

Further, the polymers of the invention may include homopolymers of one or more polymerizable compounds having three or more reactive functional groups and a rigid structural portion such as an alicyclic structure, a double bond, a triple bond or the like and selected from nucleic acids, amino acids, saccharides, fatty acids, terpenes, porphyrins, flavonoids, steroids, chlorogenic acids, and alkaloids.

### Seventh Embodiment

A rigid polymer according to the seventh embodiment of the invention will be described. The rigid polymer is a polymer obtained by using coffee bean extraction substance. Herein, as the coffee bean, Java Robusta coffee bean is used. The production of the rigid polymer is same as the production method according to the sixth embodiment.

The solubility of the polymer obtained by using the coffee bean extraction substance in respective solvents differs from that of the monomer as shown in Table 8, so that it can be understood that the polymerization is carried out.

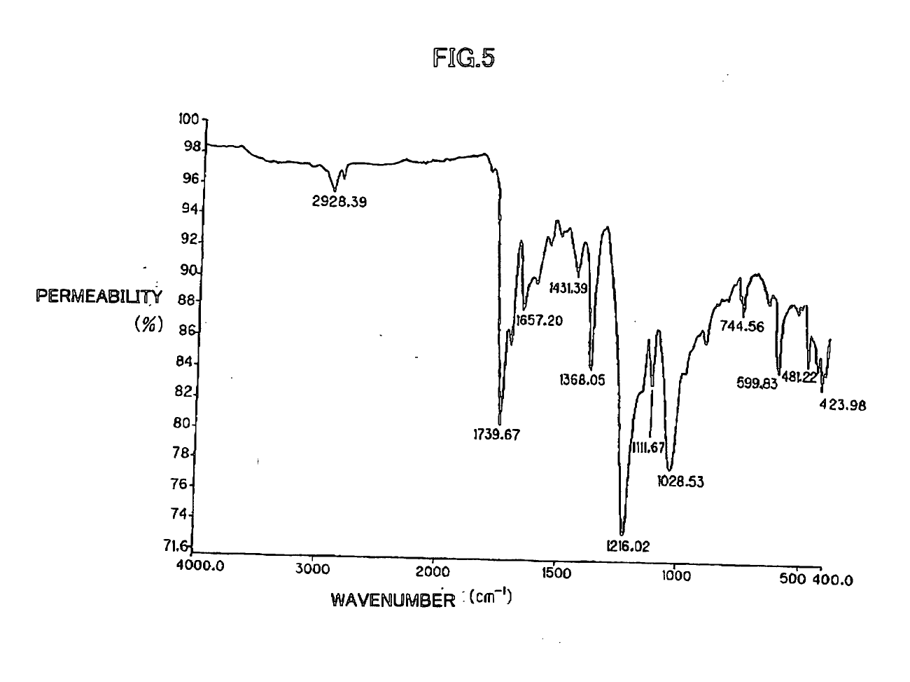

FIG. 5 shows the structure analysis result of the polymer of coffee bean extraction substance by Fourier transform infrared spectrophotometer. Since peaks attributed to aromatic rings are confirmed according to the result, it is supposed that the polymer is obtained by using coffee bean extraction substance.

### Effects of the Seventh Embodiment

According to the seventh embodiment, rigid polymer excellent in solubility in solvents and processability to be formed into thin films can be obtained by reaction of the coffee bean extraction substance.

### Eighth Embodiment

A rigid polymer according to the eighth embodiment of the invention will be described. The rigid polymer is a polymer of polylactic acid (PLA) and 4HCA. The polymer includes, as a main product, a copolymer of PLA and 4HCA and, as a by-product, homopolymers of PLA and 4HCA, respectively.

The rigid polymer is obtained by loading polylactic acid and 4HCA at each 50 wt. % into a reaction vessel, purging the reaction vessel with nitrogen in the presence of acetic anhydride and sodium acetate, and carrying out reaction at 175° C. for 6 hours.

According to the solubility test, the solubility in respective solvents is changed from that of polylactic acid and 4HCA as shown in Table 10, so that it can be understood that reaction is carried out.

TABLE 10

| | SYNTHESIS CONDITIONS | | | | | | | | | | | |
|---|---|---|---|---|---|---|---|---|---|---|---|---|
| | | | | | | | LOADING RATIO (wt %) | | | | | |
| | | | | | | | | | | | PLA/ | |
| | | | | | | | | | | | coffee | PLA/ |
| | (MONOMER) | | | | | | | | | PLA/ | bean | chlo- |
| | | | | chlo- | | coffee bean | 4HCA/ | caffeic | | quinic | extracted | rogenic |
| | | | caffeic | rogenic | quinic | extracted | PLA | acid/PLA | | acid | substance | acid |
| SOLVENTS | 4HCA | PLA | acid | acid | acid | substance | 50/50 | 50/50 | 80/20 | 50/50 | 50/50 | 50/50 |
| WATER | ◯※ | X | ◯※ | ◯ | ◯ | X | X | X | X | X | X | X |
| METHANOL | ◯ | X | ◯ | ◯ | ◯ | X | X | X | X | Δ | Δ | X |
| ACETONE | ◯ | Δ | ◯ | ◯ | X | X | Δ | Δ | ◯ | ◯ | Δ | ◯ |
| ACETONITRILE | ◯ | ◯ | ◯ | X | X | X | Δ | ◯ | ◯ | ◯ | ◯ | ◯ |
| CHLOROFORM | X | ◯ | X | Δ | X | X | Δ | ◯ | ◯ | ◯ | ◯ | ◯ |
| TFA/DCM 1/5 V/V % | ◯ | — | ◯ | — | — | — | — | — | — | — | — | — |
| TOLUENE | X | X | X | X | X | X | X | Δ | X | ◯ | Δ | Δ |
| HEXANE | X | X | X | X | X | X | X | X | X | Δ | X | X |
| NMP | ◯ | — | ◯ | — | — | — | — | — | — | — | — | — |
| DMSO | ◯ | ◯ | ◯ | ◯ | ◯ | ◯ | Δ | ◯ | ◯ | ◯ | ◯ | ◯ |
| DMF | ◯ | ◯ | ◯ | ◯ | ◯ | ◯ | Δ | ◯ | ◯ | ◯ | ◯ | ◯ |
| THF | ◯ | ◯ | ◯ | ◯ | X | X | Δ | ◯ | ◯ | ◯ | ◯ | ◯ |

◯: soluble
Δ: partially soluble
X: insoluble
◯※: soluble when heated
—: not synthesized

### Effects of the Eighth Embodiment

According to the eighth embodiment, rigid polymer excellent in solubility and processability to be formed into thin films can be obtained by reaction of 4HCA and polylactic acid.

### Ninth Embodiment

A rigid polymer according to the ninth embodiment of the invention will be described. The rigid polymer is a polymer of polylactic acid (PLA) and caffeic acid. The polymer includes, as a main product, a copolymer of PLA and caffeic acid and, as a by-product, homopolymers of PLA and caffeic acid, respectively.

The rigid polymer is obtained by loading polylactic acid and caffeic acid monomer at each 50 wt. % into a reaction vessel, purging the reaction vessel with nitrogen in the presence of acetic anhydride and sodium acetate, and carrying out reaction at, for example, 175° C. for 6 hours. The weight average molecular weight of this case is shown in Table 11.

TABLE 11

| PLA/CAFFEIC | | | WEIGHT- |
|---|---|---|---|
| ACID LOADING | | | AVERAGE |
| RATIO | POLYMERIZATION | GPC | MOLECULAR |
| (wt %) | TEMP | SOLVENT | WEIGHT |
| 50/50 | 175° C. | CHCl3 | 5900 |

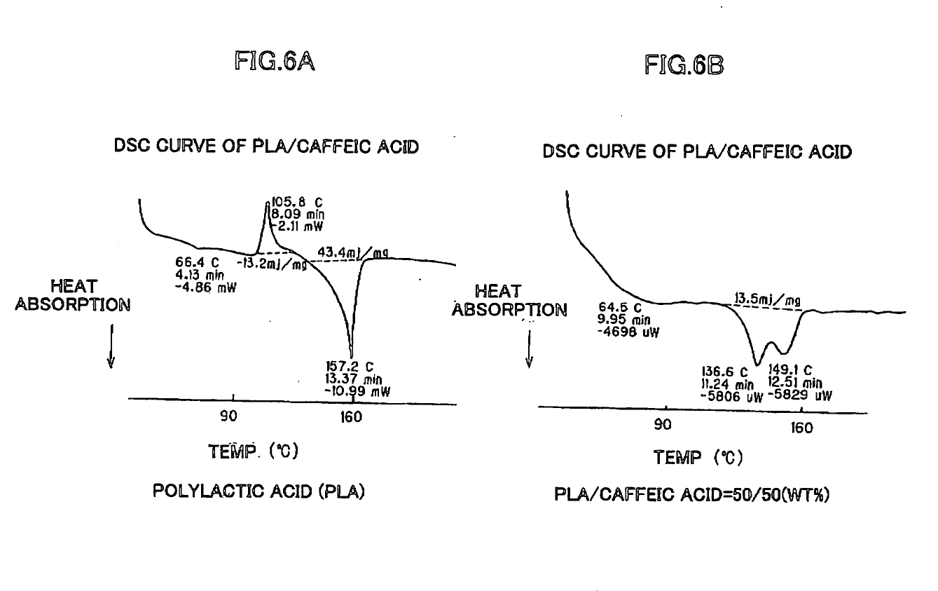

FIG. 6 shows the result of differential scanning calorimetry (DSC) of the polymer of polylactic acid and caffeic acid monomers obtained by loading the monomers each at 50 wt. %. For comparison DSC of polylactic acid is also shown. According to the result, the peak (105.8° C.) of crystallization of polylactic acid disappears. From the result, it can be assumed that another substance is polymerized in the molecular structure of polylactic acid. Consequently, it is supposed that the processability is improved.

Further, as shown in Table 10, the solubility test in respective solvents shows that the solubility is changed from that of polylactic acid and that of caffeic acid monomer.

### Effects of the Ninth Embodiment

According to the ninth embodiment, rigid polymer excellent in solubility and processability to be formed into thin films can be obtained by reaction of caffeic acid monomer and polylactic acid.

### Tenth Embodiment

A rigid polymer according to the tenth embodiment of the invention will be described. The rigid polymer is a polymer of polylactic acid (PLA) and quinic acid monomer. The polymer includes, as a main product, a copolymer of PLA and quinic acid monomer and, as a by-product, homopolymers of PLA and quinic acid monomer, respectively.

The rigid polymer is obtained by loading polylactic acid and quinic acid monomer at each 50 wt. % into a reaction vessel, purging the reaction vessel with nitrogen in the presence of acetic anhydride and sodium acetate, and carrying out reaction at, for example, 175° C. for 6 hours.

As shown in Table 10, with respect to the rigid polymer, the solubility test in respective solvents shows that the solubility is changed from that of polylactic acid and that of caffeic acid monomer.

### Effects of the Tenth Embodiment

According to the tenth embodiment, rigid polymer excellent in solubility and processability to be formed into thin films can be obtained by reaction of quinic acid monomer and polylactic acid.

### Eleventh Embodiment

A rigid polymer according to the eleventh embodiment of the invention will be described. The rigid polymer is a polymer of polylactic acid (PLA) and coffee bean extraction substance. The polymer includes, as a main product, a copolymer of PLA and coffee bean extraction substance and, as a by-product, homopolymers of PLA and coffee bean extraction substance, respectively. As the coffee bean, Java Robusta coffee bean is used.

The rigid polymer is obtained by loading polylactic acid and coffee bean extraction substance at each 50 wt. % into a reaction vessel, purging the reaction vessel with nitrogen in the presence of acetic anhydride and sodium acetate, and carrying out reaction at, for example, 175° C. for 6 hours.

As shown in Table 10, the solubility test in respective solvents shows that the solubility is changed from that of polylactic acid and that of coffee bean extraction substance monomer.

### Effects of the Eleventh Embodiment

According to the eleventh embodiment, rigid polymer excellent in solubility and processability to be formed into thin films can be obtained by reaction of monomer of coffee bean extraction substance and polylactic acid.

### Twelfth Embodiment

A rigid polymer according to the twelfth embodiment of the invention will be described. The rigid polymer is a polymer of polylactic acid (PLA) and chlorogenic acid. The polymer includes, as a main product, a copolymer of PLA and chlorogenic acid and, as a by-product, homopolymers of PLA and chlorogenic acid, respectively.

The rigid polymer is obtained by loading polylactic acid and chlorogenic acid at each 50 wt. % into a reaction vessel, purging the reaction vessel with nitrogen in the presence of acetic anhydride and sodium acetate, and carrying out reaction at, for example, 175° C. for 6 hours.

As shown in Table 10, the solubility test in respective solvents shows that the solubility is changed from that of polylactic acid and that of chlorogenic acid monomer.

### (Effects of the Twelfth Embodiment

According to the twelfth embodiment, rigid polymer excellent in solubility and processability to be formed into thin films can be obtained by reaction of chlorogenic acid monomer and polylactic acid.

The rigid polymers of the invention are not limited to the above-exemplified materials and may include rigid polymers in accordance with the required conditions and can be used as various engineering plastics. Additionally, the rigid polymers can be used, being mixed with an inorganic substance such as glass and carbon or an organic substance such as cellulose as a filler.

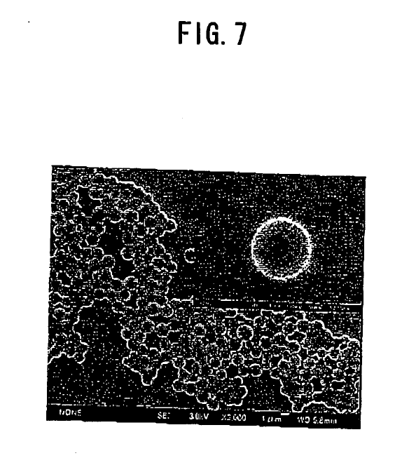

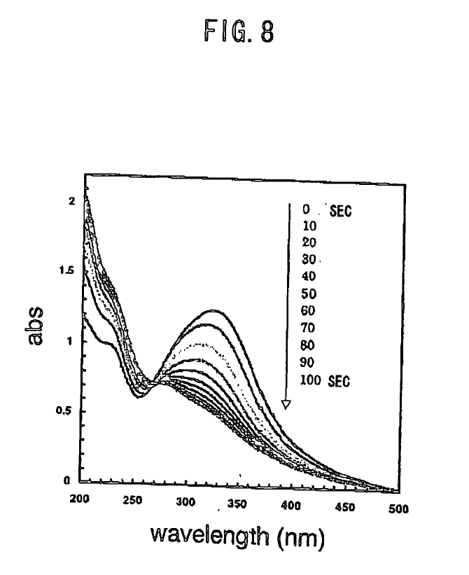

Also, it is possible to produce fine particles by inducing self-structuralization by solvent conversion utilizing the difference of the solubility of the polymers in different types of solvents. For example, FIG. 7 shows a scanning electronic microscopic photograph of fine particles of a copolymer (caffeic acid composition ratio 50%) obtained by dissolving the copolymer in dimethylformamide and adding several droplets of water to the resulting solution. From the photograph, spherical particles with about 1 μm diameter are clearly observed. Further, as shown in FIG. 8, when light (250 nm or longer wavelength) is irradiated to a thin film of the copolymer (caffeic acid 50%), that the original characteristic of the thin film of absorbing light with wavelength in a range of 250 nm to 400 nm is gradually lost with the lapse of time can be observed. It is attributed to that the structure of the copolymer is changed by light irradiation. Utilizing this characteristic, the quality improvement and decomposition property of the polymer can be controlled by light irradiation.

Further, the polymers themselves may be used as additives such as fillers and not only polylactic acid but also various kinds of polymers can be changed in the dynamic physical properties and thermal properties.

As applications of the polymers for parts, the polymers can be used for parts in various fields. For example, in fields of automotive parts, the polymers may be used, as interior parts, for a pillar garnish, a glove box, and a switch panel and as exterior parts, for a rear garnish, a side lace, a door mirror stay and as peripheral parts of an engine, a cylinder head cover, an intake manifold, a radiator, etc.

In fields of electronic/electric parts, the polymers may be used for mechanisms of VTR deck, enclosures of personal computers; keyboard stays, toner cartridges, electric refrigerators (door handles, inner boxes), room air conditioners (housings, grills, dampers), electric launders (control panels), electric vacuum cleaners (housings, suction ports), electronic microwave ovens, humidifiers, and housings of dish washers.

In fields of housing construction and accommodation facilities, the polymers may be used for door rollers for sashes, sashes, door grips, handrails, furniture parts, shower heads, ducts for air conditioners, toilet stools, unit bath members, decorative panels, wash stands, or the like.

In fields of daily utensils and goods, the polymers may be used for fasteners, tooth brushes, pen cases, cushion materials, or the like.

In fields of machines and tools, the polymers may be used for fans of motors, conveyer parts or the like.

Although the invention has been described with respect to the specific embodiments for complete and clear disclosure, the appended claims are not to be thus limited but are to be construed as embodying all modifications and alternative constructions that may occur to one skilled in the art which fairly fall within the basic teaching herein set forth.

What is claimed is:

1. A rigid polymer, comprising:
a homopolymer of one of a polymerizable bio-derived rigid compound having two or more reactive functional groups, a derivative of the bio-derived rigid compound, a hydrolyzed substance of the bio-derived rigid compound, a derivative of the hydrolyzed substance of the bio-derived rigid compound and the substances extracted from the bio resources; or
copolymers of two or more of these compound, derivatives, and substances.

2. The rigid polymer according to claim 1, wherein:
the copolymer comprises one of chlorogenic acids, chlorogenic acid derivatives, substances of hydrolyzed chlorogenic acids, derivatives of the hydrolyzed substances and substances extracted from bio-resources.

3. The rigid polymer according to claim 1, wherein:
the homopolymer comprises one of chlorogenic acids, chlorogenic acid derivatives, substances of hydrolyzed chlorogenic acids, derivatives of the hydrolyzed substances and substances extracted from bio-resources.

4. The rigid polymer according to claim 3, wherein;
the homopolymer comprises one of caffeic acid and coffee bean extracts.

5. The rigid polymer according to claim 1, wherein:
the copolymer comprises one of chlorogenic acids, chlorogenic acid derivatives, substances of hydrolyzed chlorogenic acids, derivatives of the hydrolyzed substances and substances extracted from bio-resources, and 4-hydroxycinnanic acid.

6. The rigid polymer according to claim 1, wherein:
the copolymer comprises one of chlorogenic acid, caffeic acid, quinic acid and coffee bean extracts, and 4-hydroxycinnanic acid.

7. The rigid polymer according to claim 6, wherein:
the coffee bean extracts are those obtained by extraction of Robusta coffee bean.

8. The rigid polymer according to claim 1, wherein:
the copolymer comprises one of chlorogenic acids, chlorogenic acid derivatives, the substances of the hydrolyzed chlorogenic acids, derivatives of the hydrolyzed substances and substances extracted from bio-resources, and polylactic acid.

9. The rigid polymer according to claim 8, wherein:
the copolymer comprises one of chlorogenic acid, caffeic acid, quinic acid, 4-hydroxycinnanic acid and coffee bean extracts, and polylactic acid.

10. The rigid polymer according to claim 1, wherein:
the homopolymer or the copolymer has at least one rigid structural portion of a polymerizable aromatic ring having two or more reactive functional groups, a polymerizable alicyclic ring having two or more reactive functional groups, a polymerizable double bond having two or more reactive functional groups, or a polymerizable triple bond having two or more reactive functional groups.

11. The rigid polymer according to any one of claims 1 to 10 soluble in a solvent and formable to be a thin film or fine particles.

12. A production method of a rigid polymer comprising one of a bio-derived rigid compound, a derivative of the bio-derived rigid compound, a hydrolyzed substance of the bio-derived rigid compound, a derivative of the hydrolyzed substance, or a substance extracted from a bio-resource, wherein the method comprises steps of:
acetylating one of the polymerizable bio-derived rigid compound having two or more reactive functional groups, the derivative of the bio-derived rigid compound having two or more reactive functional groups, the hydrolyzed substance of the bio-derived rigid compound having two or more reactive functional groups, the derivative of the hydrolyzed substance having two or more reactive functional groups and the substance extracted from the bio-resource having two or more reactive functional groups; and
homo-polymerizing the acetylated compound or substance in the presence of a prescribed catalyst.

13. The production method according to claim 12, wherein;
the bio-derived rigid compound or the hydrolyzed substance is one of chlorogenic acid, caffeic acid, quinic acid and a coffee extract.

14. A production method of a rigid polymer by copolymerizing one of a first bio-derived rigid compound, a derivative of the first bio-derived rigid compound, a hydrolyzed substance of the first bio-derived rigid compound, a derivative of the hydrolyzed substance of the first bio-derived rigid compound and a first substance extracted from a bio-resource with one of a second bio-derived rigid compound, a derivative of the second bio-derived rigid compound, a hydrolyzed substance of the second bio-derived rigid compound, a derivative of the hydrolyzed substance of the second bio-derived rigid compound and a second substance extracted from a bio-resource, wherein the production method comprises steps of:
acetylating a first material comprising one of the polymerizable first bio-derived rigid compound having two or more reactive functional groups, the derivative of the first bio-derived rigid compound, the hydrolyzed substance of the first bio-derived rigid compound, the derivative of the hydrolyzed substance of the first bio-derived rigid compound and the first substance extracted from the bio-resource as well as a second material comprising one of the polymerizable second bio-derived rigid compound having two or more reactive functional groups, the derivative of the second bio-derived rigid compound, the hydrolyzed substance of the second bio-derived rigid compound, the derivative of the hydrolyzed substance of the second bio-derived rigid compound and the second substance extracted from the bio-resource; and
copolymerizing the first acetylated material with the second acetylated material in the presence of a prescribed catalyst.

15. The production method according to claim 14, wherein:
the first bio-derived rigid compound is 4-hydroxycinnamic acid, and
the second bio-derived rigid compound or its hydrolyzed substance is one of chlorogenic acid, caffeic acid, quinic acid and a coffee extract.

16. The production method according to claim 12 or 14,
wherein:
acetic anhydride and trifluoroacetic anhydride are used as a acetylation agent.
